(19) United States
(12) Patent Application Publication
(10) Pub. No.: US 2011/0301642 A1
(43) Pub. Date: Dec. 8, 2011

(54) Curved needle

(76) Inventors: Geoffrey H. White, Birchgrove, NSW (AU); Paul Gilson, Uggool, Moycullen, Co. Galway (IE)

(21) Appl. No.: 13/128,561

(22) PCT Filed: Nov. 13, 2009
(86) PCT No.: PCT/AU2009/001476

§ 371 Date: Aug. 26, 2011

(30) Foreign Application Priority Data
Nov. 13, 2008 (AU) 2008905868

(51) Int. Cl. A61B 17/06

(52) U.S. Cl. 606/223

(57) ABSTRACT

A needle assembly and method for ligating a vessel. The needle shaft is substantially curved and carries a shuttle tethered to a needle. The shuttle is configured to move longitudinally between the ends of the curved shaft during use.

### FIELD OF THE INVENTION

The present invention relates to needles and particularly to a curved medical needle assembly.

### BACKGROUND ART

Many types of needles are available and for a variety of surgical indications, ranging from simple closure of surface wounds to complex repair of parts of the vasculature or other tissues.

In a most basic form, sutures are attached to a needle through an eye at the end of the needle or are attached directly to the end of the needle by, for example, crimping. Following puncture through tissue and tying of an end, the needle travels subcutaneously to an exit point whereupon both the needle and trailing suture are pulled through the tissue. The suture is tied at the exit point and the steps repeated as required.

In subcutaneous ligation, repair or modification of vascular or other structures, a conventional needle/suture structure is used to enter via the skin and exit at a spaced point to bring the suture into close proximity with the structure. The process of entering and exiting the skin multiple times tethers sections of the skin and subcutaneous fascia and draws them downwards and together in such a way that the skin becomes deformed or torn and the suture cannot be approximated sufficiently to provide closure ligation of the structure.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In a first aspect, the present invention consists in a needle assembly comprising:
a curved shaft having a first tissue piercing end and a second tissue piercing end;
at least one shuttle member moveable relative to said shaft between said first and second tissue piercing ends;
said at least one shuttle member comprising at least one suture securing means to secure said at least one suture to the shuttle member.

By the term “suture”, it is to be understood that the scope of this invention covers various forms of suture including tape, thread and cord. The suture may be made from an absorbable material such that it is resorbed in situ. Alternatively, the suture may be made from a non-resorbable material. In one embodiment, the suture may be made from a metallic material. The suture may further have elastomeric properties as discussed in more detail below. The suture may also be made of a combination of materials.

According to a second aspect, the present invention is a method of ligating, repairing or modifying a target structure within an individual, comprising:
(a) providing a needle assembly comprising a curved shaft having a first tissue piercing end and a second tissue piercing end;
a shuttle member moveable relative to said curved shaft between said first and second tissue piercing ends;
said shuttle member comprising at least one suture securing means which secures at least one suture to the shuttle member;
(b) at a primary access site in the skin, directing the first tissue piercing end of the curved shaft to a position adjacent to the target structure;
(c) moving said curved shaft in a first direction such that said first tissue piercing end and at least a leading portion of the suture passes superiorly to or through said target structure and to a location beyond said target structure;
(d) causing said first tissue piercing end to exit the skin at a first exit site remote from the primary access site and wherein the second tissue piercing end, the shuttle member and at least a portion of the at least one suture is retained subcutaneously;
(e) continuing movement of the curved shaft further in said first direction such that the second tissue piercing end is also positioned beyond said target structure;
(f) exerting pressure on said needle assembly to move the second tissue piercing end and the at least said leading portion of the suture in a second, different direction such that said second tissue piercing end and the at least leading portion of the suture passes inferiorly to or through said target structure;
(g) causing said second tissue piercing end to exit the skin at a second exit site remote from the primary access site and the first exit site wherein the first tissue piercing end, the shuttle member and at least a portion of the at least one suture is retained subcutaneously;
(h) securing at least the leading portion of the suture to a trailing portion of the suture.

According to a third aspect, the present invention is a method of ligating, repairing or modifying a target structure within an individual, comprising:
(a) providing a needle assembly comprising a curved shaft having a first tissue piercing end and a second tissue piercing end;
a shuttle member moveable relative to said curved shaft between said first and second tissue piercing ends;
said shuttle member comprising at least one suture securing means which secures at least one suture to the shuttle member;
(b) at a primary access site in the skin, directing the first tissue piercing end of the curved shaft to a position adjacent to the target structure;
(c) moving said curved shaft in a first direction such that said first tissue piercing end and at least a leading portion of the suture passes inferiorly relative to said target structure and to a location beyond said target structure;
(d) causing said first tissue piercing end to exit the skin at a first exit site remote from the primary access site and wherein the second tissue piercing end, the shuttle member and at least a portion of the at least one suture is retained subcutaneously;
(e) continuing movement of the curved shaft further in said first direction such that the second tissue piercing end is also positioned beyond said target structure;
(f) exerting pressure on said needle assembly to move the second tissue piercing end and the at least said leading portion of the suture in a second, different direction such that said second tissue piercing end and the at least leading portion of the suture passes superiorly relative to said target structure;
(g) causing said second tissue piercing end to exit the skin at a second exit site remote from the primary access site and the first exit site wherein the first tissue piercing end, the shuttle member and at least a portion of the at least one suture is retained subcutaneously;
(h) securing at least the leading portion of the suture to a trailing portion of the suture.

In one embodiment of the second and third aspects, step (h) may comprise exerting pressure on the needle assembly to move the first tissue piercing end and the at least said leading portion of the suture in a third direction such that said first tissue piercing end and the at least leading portion of the suture pass through the primary access site. In an embodiment wherein the structure is to be ligated, the suture may then be secured and tightened around the structure to ligate said structure. Alternatively, the suture may be secured to only partially ligate said target structure.

The terms “inferior” and “superior” are not intended to be afforded their medical definition but rather provide a spatial definition relative to the target vessel or structure.

In a further aspect, the present invention provides a needle assembly comprising:
a curved shaft having a first tissue piercing end and a second tissue piercing end;
at least one shuttle member moveable relative to said shaft between said first and second tissue piercing ends; and
at least one suture connected to said at least one shuttle member.

The structure may comprise a vessel. The vessel may comprise a vein. Alternatively the vessel may comprise an artery. The structure may comprise non-vascular structures. The structure may further comprise ducts. An example includes bile ducts. In a′ further embodiment, the structure may comprise a shunt or bypass graft either made from the patient's own tissue or from artificial materials.

The needle assembly of the present invention may also be used to draw together other tissue structures such as divided muscle, for example, bringing together divided muscle and fascial layers in a hernia repair or for drawing tissues closer or supporting tissue such as in plastic surgery procedures. In one embodiment, the needle assembly has application in endoscopic procedures. In these embodiment, the needle is not actually piercing the skin but rather passing through other tissues. Therefore, the term “tissue piercing” as herein described should be read to include said other tissues. The shaft of the needle assembly may comprise or include a circular arc. The curvature of the shaft may vary and may subtend an arc anywhere between approximately 90° and 300°. Preferably, the shaft subtends an arc between 100° and 150°, more preferably between 110° and 130°, and more preferably approximately 120°.

Further, in another embodiment, the curvature of the shaft may be non-circular. For example, the curvature may be elliptical, parabolic or hyperbolic. In one embodiment, the curve may comprise a combination of these curves,

The shaft may further include one or more non-curved sections. In this embodiment said non-curved sections may be positioned adjacent to one or both ends of the shaft. The shaft may further comprise non-planar, three dimensional configurations.

The cross-section of the curved shaft may be circular. In a further embodiment the cross section of the curved shaft may be non-circular. The cross section of the curved shaft may be oval or elliptical, or flattened or triangular.

The shuttle member is preferably longitudinally moveable along at least along a length of said curved shaft. The shuttle member may also be rotationally moveable relative to the shaft. In a preferred embodiment said shuttle member is fully rotatable relative to the shaft.

Preferably, the shuttle member is positioned external to the shaft. The shuttle member may, alternatively, be positioned at least partially within the shaft. In the latter embodiment, at least part of the shaft is tubular, with an internal lumen to receive at least part of the shuttle.

In the embodiment wherein the shuttle is external to the shaft, said shaft is preferably a solid member. The shuttle member may comprise a substantially tubular body extending from a proximal end to a distal end and having an inner wall defining a lumen to receive at least part of the shaft. The entire inner wall of the shuttle member may interface with the external surface of the shaft. The interface between the shuttle member and the shaft is such that the shuttle is longitudinally moveable along a length of the shaft.

There may be some degree of friction between the shuttle member and the curved shaft irrespective of whether the shuttle is internal or external the shaft. This friction does not prevent said longitudinal movement of the shuttle member relative to the curved shaft.

The degree of friction between the shuttle member and the curved shaft may vary and in one embodiment, the friction may vary along the length of the curved shaft. In this embodiment, there may be an area of increased friction between said two components to cause the shuttle to slow and in some embodiments stop.

In the embodiment of the invention wherein the shuttle member is external the curved shaft, the increase in friction may be realised by an increase in diameter of the curved shaft to bring the shuttle member and curved shaft into relatively tight frictional engagement. In another embodiment, the friction may be increased by providing a different material and/or surface features of the curved shaft at or in a region along its length. Preferably, the curved shaft has at least two areas of increased frictional engagement with the shuttle member. Said areas of increased friction may be substantially adjacent to the first and second tissue piercing ends so as to prevent the shuttle member from travelling beyond said ends.

The needle assembly may also comprise at least one stop member on said curved shaft. Preferably, the assembly includes at least two stop members relatively spaced on the outer surface of the shaft. The stop members stop the longitudinal travel of the shuttle and so are preferably positioned adjacent to the first and second tissue piercing tips respectively. In another embodiment, the stop members may be more closely spaced to one another to limit the longitudinal movement of the shuttle.

The stop members typically comprise ramp-like structures. In this embodiment; the proximal or distal ends of the shuttle ride up the ramp until the shuttle is prevented from moving further in a longitudinal direction. When the shaft is moved in a different direction, the shuttle slides back down the ramp and travels longitudinally along said shaft. The ramp-like structure aids in preventing “sticking” of the shuttle on the stop member.

In one embodiment, only the inner wall at or adjacent to the distal end and/or the proximal end of the shuttle engages the external surface of the shaft.

The inner wall and/or outer wall of the shuttle member may be substantially straight along a longitudinal axis. Alternatively, the inner and/or outer walls may curve between the proximal and distal ends thereof. The inner wall and/or outer wall may curve convexly or concavely between said proximal and distal ends. Typically, the shuttle member comprises an inner concavely curving wall and an outer convexly curving wall. Alternatively, the inner wall may be substantially straight and the outer wall convex. The outer wall may be substantially straight and the inner wall concave.

The inner cross section of the shuttle member preferably corresponds to the outer cross section of the needle shaft.

Preferably, the outer surface of the shuttle member is relatively smooth with no sharp edges or angles. At the proximal end of the shuttle member, the inner and outer surfaces of the shuttle are spaced by a leading end face. In this embodiment, both the inner and outer surfaces may taper at a joining region between the inner and outer surface and the leading end face. The leading end face may be substantially planar. Alternatively, the leading end face may comprise a substantially domed surface.

Similarly, the distal end may comprise a trailing end face wherein the inner and outer surfaces taper at a joining region between the internal and outer surface and the trailing end face. The trailing end face may be substantially planar. Alternatively, the trailing end face may comprise a substantially convex or concave domed surface.

The cross-sectional shape of the shuttle member preferably corresponds to the cross-sectional shape of the curved shaft.

The shuttle member of the invention is typically manufactured as one piece. The shuttle member may be made from any suitable biocompatible material. Preferably it is made from a metal or a metal alloy including stainless steel, nickel, aluminium, titanium, zirconium, niobium, molybdenum, silver, indium, hafnium, tantalum, tungsten, iridium, platinum and gold, copper or alloys of said elements. Alternatively, the shuttle member may be made from a non-metallic material including one or more biocompatible polymers or ceramics.

The shuttle member may further comprise a coating. The coating may include a lubricious coating. Examples of suitable coatings include silicones or polytetrafluoroethylene (PTFE).

In embodiments wherein the shuttle member is made from a non-radiopaque material, it may include radiopaque markers to allow x-ray/fluoroscopic visualisation by a surgeon during a surgical procedure.

Preferably the curved shaft and/or shuttle are made from a material visible on external ultrasound imaging, or include markers which enhance its visibility to ultrasound imaging, to allow visualisation of the procedure by ultrasound monitoring. Enhancement of visualisation may be achieved by modifying the surface of the shuttle member or parts or all of the shaft. Surface modifications, may include any combination or single incorporation of any one of knurling, longitudinal scoring, circumferential scoring, sand grit or bead blasting, chemical or electrochemical etching or equivalent surface modification process.

The suture securing means of the shuttle may comprise at least one aperture in the shuttle. The aperture may extend from a first opening defined by the outer wall to a second opening defined by the inner wall. The suture securing means may further comprise a fixing member to fix a suture in said at least one aperture. A plurality of sutures may be secured to the shuttle member.

Alternatively, the suture securing means may comprise at least two apertures in the shuttle. In this embodiment, both of said apertures extend from respective first openings defined by the outer wall to respective second openings defined by the inner wall. Said at least two apertures may be located relatively adjacent to each other along substantially the same longitudinal axis of the shuttle member. Similarly, they two apertures may be positioned relatively adjacent to each other along substantially the same lateral axis.

A leading length of the suture may be threaded through one of said apertures and out of the other aperture to secure it to the shuttle member. The leading length of the suture may be secured to a trailing length of the suture by, for example, tying, crimping, adhesive, welding, clipping or other such means. The result is a closed loop of suture secured to the shuttle member via said apertures.

The suture securing means may further comprise at least one tethering member. The tethering member may be spaced from said shuttle member and may tether the suture to the shuttle member. In one embodiment, a leading length of a suture is threaded through the one aperture and out another aperture in the shuttle. The leading length is tethered to a trailing length of the suture by said tethering member. In a further embodiment, the suture securing means comprises multiple tethering members.

Alternatively, the shuttle member may be joined to the suture or tether by way of a universal joint. In one embodiment the joint may include a ball and socket joint. Alternatively, the joint may include a hinge arrangement. The universal joint may facilitate the change in direction of the shuttle member during use.

The at least one tethering member typically comprises a tubular body having an inner lumen to receive a length, of suture. The inner lumen is typically substantially straight. The outer surface of the tethering member may be straight or curved. Wherein the outer surface is curved, it may be curved either convexly or concavely. In the latter configuration, the tethering member may comprise a dumbbell structure with first and second end regions of the tubular body having a greater thickness than a relatively thinner central crimping region.

While the forgoing description relates to a single suture directly or indirectly attached to an internal or external shuttle member, the scope of the present invention includes multiple attachments. In one embodiment, a shuttle member may comprise multiple suture securing means to secure multiple sutures thereto. Alternatively, the assembly may comprise multiple shuttle members each comprising a single suture securing means for a single suture. In a further embodiment, the assembly may comprise multiple shuttle members each comprising multiple attachment means.

In an embodiment wherein the assembly includes multiple shuttle members, the shuttle members may be configured to act in concert. Alternatively, the shuttle member may move independently of each other.

In a further embodiment, the assembly may comprise one or more magnetic shuttles. Movement of the magnetic shuttle may be achieved by application of magnetic forces from an external source. Alternatively, to allow for longitudinal displacement of the shuttle member, the assembly may comprise an internal, flexible drive member that engages the shuttle member. The drive member may move the shuttle member by way of a torque applied to the drive member which results in linear movement of the shuttle member and suture along a desired path.

The tethering member is typically made from a biocompatible material. The biocompatible material may include a metal or alloy. Preferably, the material is substantially malleable to allow the tethering member to be readily crimped around the suture. Examples of suitable materials include stainless steel, nickel, aluminium, titanium, zirconium, niobium, molybdenum, silver, indium, hafnium, tantalum, tungsten, iridium, platinum and gold, copper or alloys of said elements. Alternatively, the tethering member may be made from a non-metallic material including one or more biocompatible polymers. In this embodiment, said biocompatible polymer may include radiopaque markers thereon or therein.

If using a metallic or other tethering member with surface modification, radiopaque or ultrasound markers, a user may visualise the tethering member thus enabling precise control during a suturing procedure.

The tissue piercing ends of the curved shaft may comprise a number of different types of tips. An example of a suitable tip is a trocar tip.

The curved shaft is preferably made from a suitable biocompatible material including metals and metal alloys or a combination thereof. In one embodiment, the curved shaft is made from stainless steel. The stainless steel may include surgical grades 316 and 420. Further, the curved shaft may be made from a suitable polymeric material. In another embodiment, the curved shaft may be made from a combination of materials. One example is a combination of a suitable metal and a polymeric material.

The curved shaft and/or the shuttle and/or the tethering member may be machine polished, or eleetro-polished post manufacture such that the surfaces are free from scratches and burrs. One or more component may also be coated with a lubricious coating. Examples of suitable coating materials include silicones and polytetrafluoroethylene (PTFE).

In certain embodiments, the suture thread used with the needle assembly of the present invention may comprise an elastomeric structure. The entire suture thread may be made from an elastomeric material. Alternatively the thread may have an elastomeric core which is overlayed by a sheath. The sheath may have less elasticity than the core. Alternatively the sheath may have greater elasticity than the core. Alternatively, the core may be metallic.

The entire thread may be made from a suitable biocompatible material. In embodiments comprising a core and a sheath, the sheath may be made from a suitable biocompatible material.

Alternatively, the sheath may also be made from an elastomeric, material, different to the elastomeric material of the core. For example, in this embodiment, while still having elastomeric properties, the sheath may not be stretched to the same extent as the core. It is preferable however, that the sheath or other interface with the body is made from a relatively biocompatible material.

The material of the suture thread may be a polymeric-based material. Examples of suitable materials include silicone rubber. In further embodiments, the elastomeric material may comprise purified natural rubber, isoprene cisobutylene, abrylonitrile butadiene copolymer or derivatives.

The sheath of the suture thread may include polytetrafluoroethylene (PTFE), polyvinylidine fluoride, polypropylene, polyurethane-polyether, collagen, polyglyconate or derivatives. Natural fibres which may be used include silk, collagen, cotton and linen.

The sheath may be helically wound around the core. Alternatively, the sheath may be, interwoven to form a mesh structure around the core. Further, the sheath may be molded with the core to form a unitary structure.

The suture thread, whether having elastic properties or not may further comprise pharmacological substances within its structure for release into the surrounding tissue when implanted in a subject. For example, the thread may include or be coated with anti-inflammatory agents or antibiotics. In still further embodiments, the thread may include agents to enhance tissue ingrowth. The thread may further be coated with agents to improve lubricity, including silicone or polytetrafluoroethylene (PTFE).

The suture thread may be substantially circular in cross-section. Alternatively the suture thread may be relatively flattened. In one embodiment the cross-section could be oval or elliptical in cross-section. In a further embodiment, the cross section may be star shaped or trefoil.

The suture may be delivered in a spiral or helical configuration. Preferably, in embodiments wherein the suture thread is at least partially elastomeric it may stretch by up to 100% of its length. In other embodiments, the suture thread is capable of stretching by up to 75% of its length, or up to 50% of its length or up to 25% of its length. In one embodiment, the suture thread may stretch by between 5% and 25% of its length.

The suture thread, whether having elastic properties or not, may also include one or more barbs along its length. The barbs are preferably molded on the thread such that the risk of dislodgement in situ is minimised.

The barbed suture may be configured as a single-ended suture with a plurality of barbs aligned to allow the suture to move through tissue in one direction and to resist moving through the tissue in the other direction. The barbed suture may also comprise a double-ended suture wherein the barbs on a first end portion are arranged to facilitate the suture to move through tissue in a first direction and the barbs on a second end portion are aligned to allow the suture to move through tissue in a second different direction.

The barbs may be closely spaced along the suture body for situations where a high gripping force is needed. Alternatively they may be spaced apart for applications where less gripping is required.

The barbs may include one or more extension legs. The legs of each barb may be oriented in the same direction along the length of the thread. The legs may be substantially straight or may have a curved surface to allow the suture thread to smoothly pass through tissue.

The barbs may be integrally molded with the suture thread. Alternatively the barbs may be attached to the thread. The barbs may be made from the same or a different material to the suture thread.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

### BRIEF DESCRIPTION OF THE DRAWINGS

FIG. 1 is a schematic representation of the assembly of the present invention;

FIG. 2 is an exploded cross-sectional view of the shuttle component of the assembly of FIG. 1;

FIG. 3 is a side elevational view of a shuttle according to the present invention;

FIG. 3a is a sectional view of the shuttle of FIG. 3;

FIG. 3b is a cross sectional view of FIG. 3a;

FIG. 4 is a schematic view of the shuttle and shaft with ramp members;

FIG. 5 is a sectional view of the shuttle, suture and shaft of an embodiment of the present invention;

FIG. 6 is a side elevational view of a curved shaft of an embodiment of the present invention;

FIGS. 7, 7a and 7b depict an embodiment of the tethering member of the present invention;

FIGS. 8a to 8d are schematic representation of a ligation procedure of a vessel using the needle assembly of the present invention;

FIGS. 9a and 9b represent the path of a suture during the procedure represented in FIGS. 8a to 8d;

FIGS. 10a and 10b show alternative paths of a suture;

FIG. 11a and 11b depict ligation of a vessel at the end of a procedure; and

FIG. 12 depicts the calf area of an individual following a procedure as represented in FIGS. 8a to 8d.

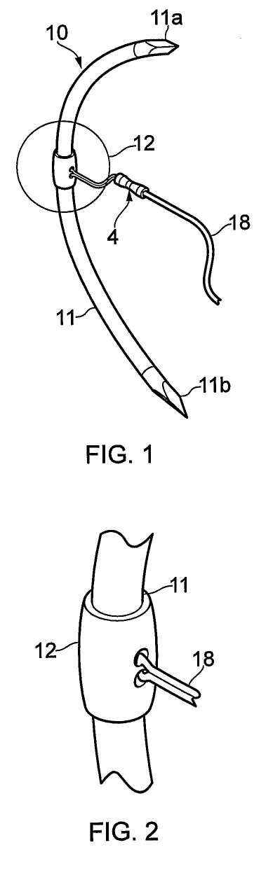

### DETAILED DESCRIPTION OF AN EXEMPLARY EMBODIMENT OF THE INVENTION

The needle assembly 10 of the present invention includes a curved shaft 11 and a shuttle 12. The shaft 11 has two tapered ends 11a and 11b which are designed to pierce tissue. Ends 11a and 11b are shown as trocar tips.

The shuttle 12 is longitudinally moveable along a length of the shaft 11 and comprises a securing means to secure a suture 18 to the shuttle 12.

The shuttle 12 in addition to being longitudinally moveable along a length of the curved shaft 11 is also fully rotatable relative to said shaft 11.

The shuttle member may, alternatively be configured such that it cannot rotate relative to the shaft. For example, the shaft may have an elliptical cross section and the shuttle an elliptical lumen therein.

In the embodiments depicted, the shuttle 12 is positioned outside the shaft 11. The shaft in these embodiments is a solid structure which provides a stiffer structure at any given diameter than a needle shaft with either an eye or having a slot to receive a shuttle 12.

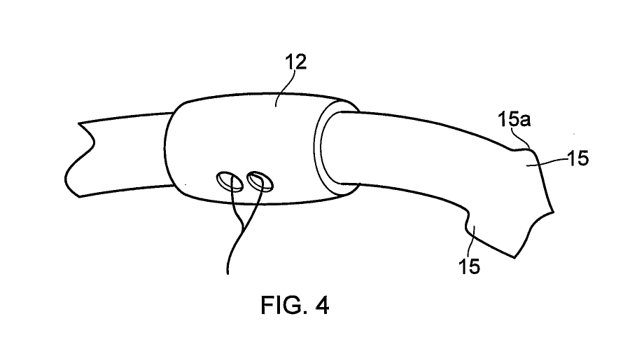

The shuttle 12 is depicted in FIG. 3 as a substantially tubular body 13 extending from a proximal end 13a to a distal end 13b. An inner wall 14 defines a lumen to receive the shaft 11 of the needle assembly 10. As mentioned above, there may be some degree of friction between the shuttle 12 and the shaft 11. The degree of friction between the shuttle 12 and the shaft 11 may vary along the length of the shaft 11. While variable, the friction must not be so great as to limit the movement of the shuttle along a substantial length of the shaft. The invention requires a relatively free longitudinal movement of the shuttle along the shaft to cause suture 18 to also follow the path of movement along the shaft. A relatively free rotation of the shuttle around the shaft is also desirable.

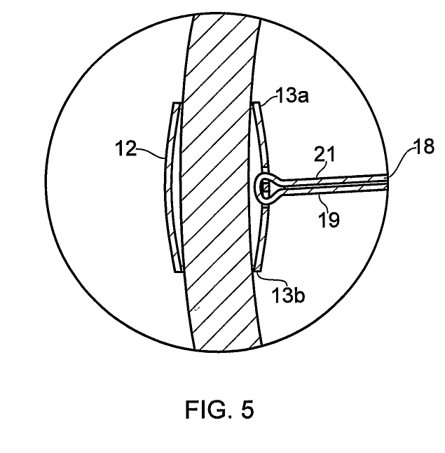

To avoid, the shuttle 12 sticking at any point along the shaft, it may be stopped by discrete stop members 15 as depicted in FIG. 4 which comprise ramp members 15. The shuttle 12 rides up the ramp until it is prevented from moving further in a longitudinal direction due to the height of the highest point of the ramp being greater than the diameter of the shuttle. When the shaft is moved by a user into a different orientation during a procedure, the shuttle may slide back down the ramp and travel longitudinally along said shaft in the opposite direction

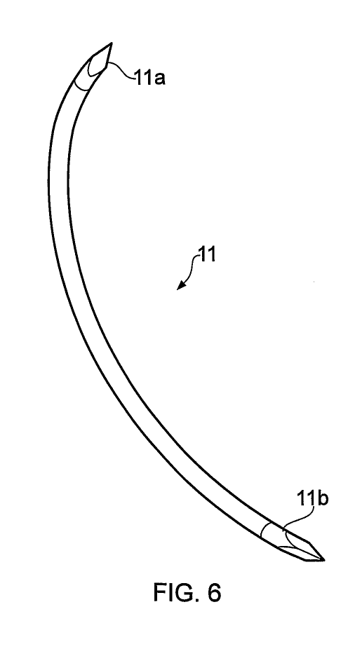

In the cross-sectional representation in FIG. 5, the shuttle is depicted as engaging the shaft 11 only at its proximal and distal ends 13a and 13b. This limits the degree of friction between the two components allowing the shuttle to move relatively freely along the length of the shaft 11.

The shuttle is shown as comprising a relatively curved tubular structure with both a curved inner wall 14 and a curved outer wall 16. The diameter of the shuttle at its greatest is desirably no greater than the cutting part of the needle or the diameter of the shaft wherein the stop members are located.

The smooth, curved outer surface of the shuttle prevents it nicking or cutting adjacent tissue as it shuttles along the shaft.

The suture securing means of the shuttle is depicted in FIG. 3a as two apertures 17a and 17b in the shuttle. Both the apertures extend from an opening defined by the outer wall 16 to a second opening defined by the inner wall 14.

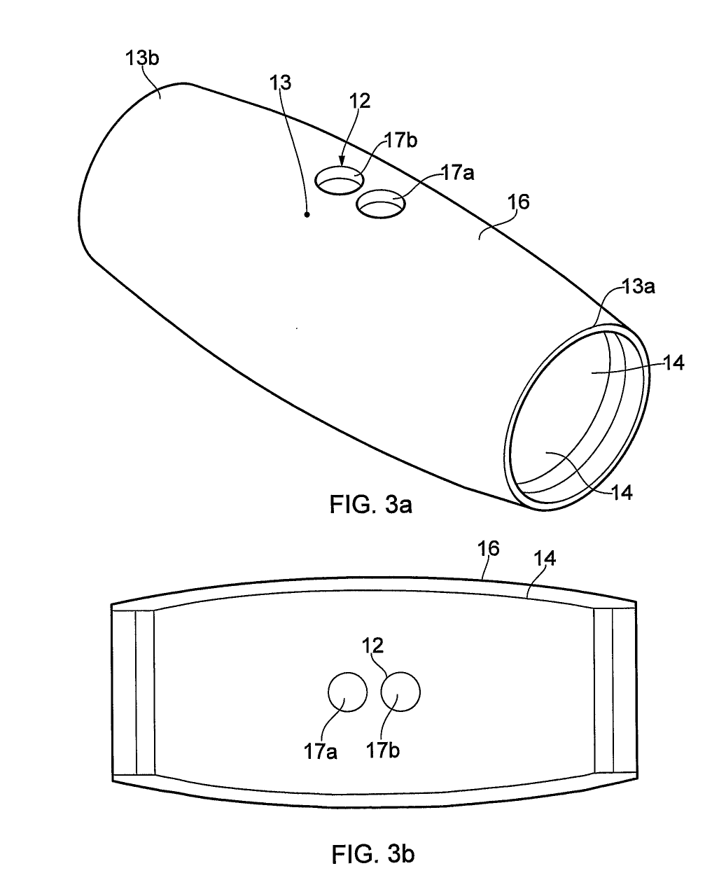

The apertures are positioned adjacent to one another such that a suture 18 may be threaded therethrough as shown in detail in FIGS. 2 and 5.

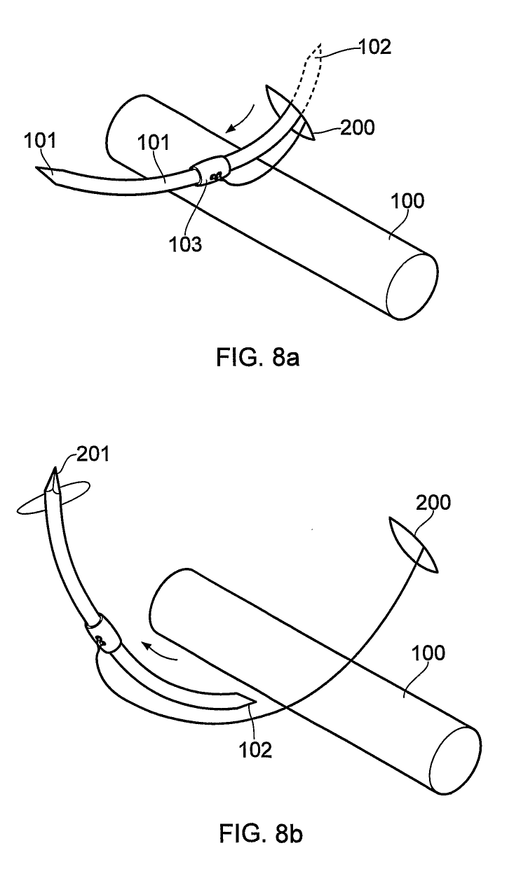

A leading length 19 of the suture is secured to a trailing length 21 of the suture by a tethering member 22. The tethering member 22 is spaced from said shuttle 12 and comprises a tubular body 23 having an inner lumen 24 to receive a length of suture 18. The inner lumen 24 of the tethering member 22 is substantially straight whereas an outer surface 25, as depicted in FIG. 7a, defines a dumbbell structure with first and second end regions 24a and 24b having a greater, thickness than a thinner central crimping region 25.

The tethering member is made from a malleable, material which may be crimped at least at region 25 around the suture.

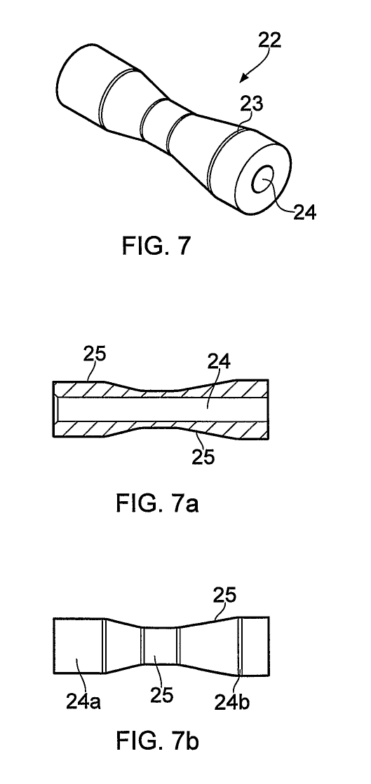

FIG. 6 depicts the curved shaft without the shuttle. The curvature of the shaft may vary and may subtend an arc anywhere between approximately 90° and 300°. In the embodiment depicted the are subtends an angle of approximately 120°.

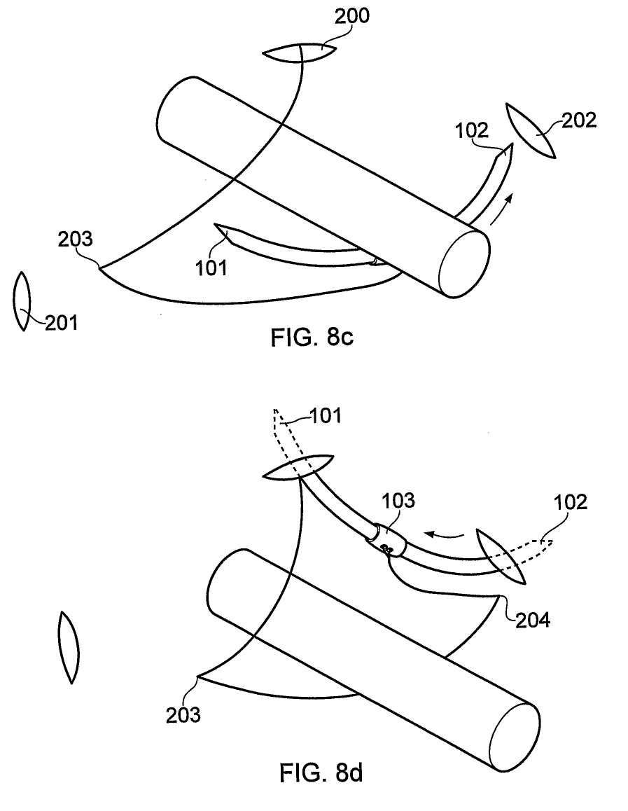

FIGS. 8a to 8d depict schematic representations of a ligation procedure. In the depicted examples, full ligation of a vessel 100 is shown although it is to be noted that partial ligation may be desirable. Similarly the technique may be used to repair and/or modify a structure such as a vessel. Structures other than vessels are discussed above. While vessel 100 is depicted as roughly parallel to the skin surface (access portal 200), it is to be understood that the vessel may have a range of orientations. Indeed in the case of perforating veins, these are more typically oriented at an angle of roughly 90° to the skin surface.

In FIG. 8a needle assembly 101 has a first end 101 and a second end 102. Shuttle 103 is slidably moveable along its length. First end 102 is introduced through access site 200 and in the embodiment shown, the assembly 100 passes superiorly relative to vessel 100 through the fatty tissue surrounding the vessel (not depicted). When first end 101 has travelled sufficiently beyond said vessel, it is caused to exit the skin via exit site 201. A surgeon can then pull this end 101 of the assembly to draw the second end 102 to a position beyond the vessel as depicted in FIG. 8b. This “frees” the second end 102 to then pass beneath the vessel in the opposite direction as shown in FIG. 8c.

Second end 102 then exits the skin at a second exit site 202 and the assembly is pulled by a surgeon to a position where the first end passes under or around the vessel and lies within the fatty tissue beneath exit site 202.

The surgeon then pivots the assembly to cause the first end 101 to pass through the fatty tissue and exit via access portal 200.

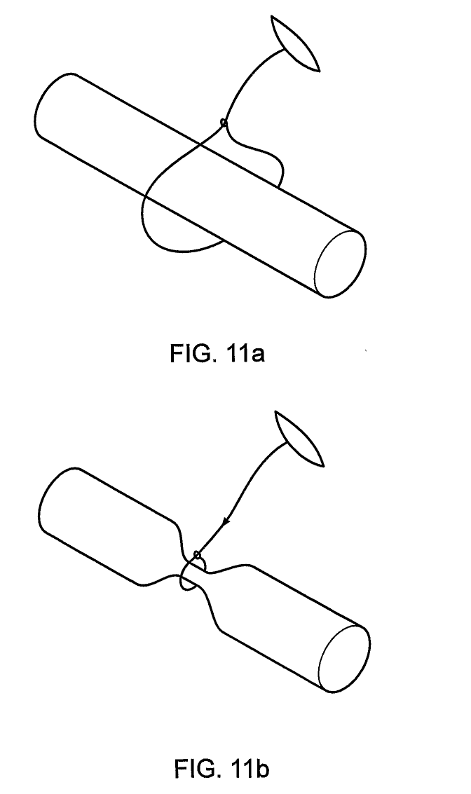

With suture 99 shuttling along the length of the assembly during the procedure, it is caused to create a loop around the vessel. The ends of the suture may then be tied and tightened around the vessel 100 as depicted in FIGS. 11a and 11b.

At regions 203 and 204 depicted in FIGS. 8c and 8d, the suture is anchored in the fatty tissue around the vessel 100. Accordingly, the suture is retained entirely within the fatty tissue plane surrounding the vessel at all times. This avoids the pulling of skin and fascia as encountered with conventional needles and sutures as they enter and exit the skin and tissue.

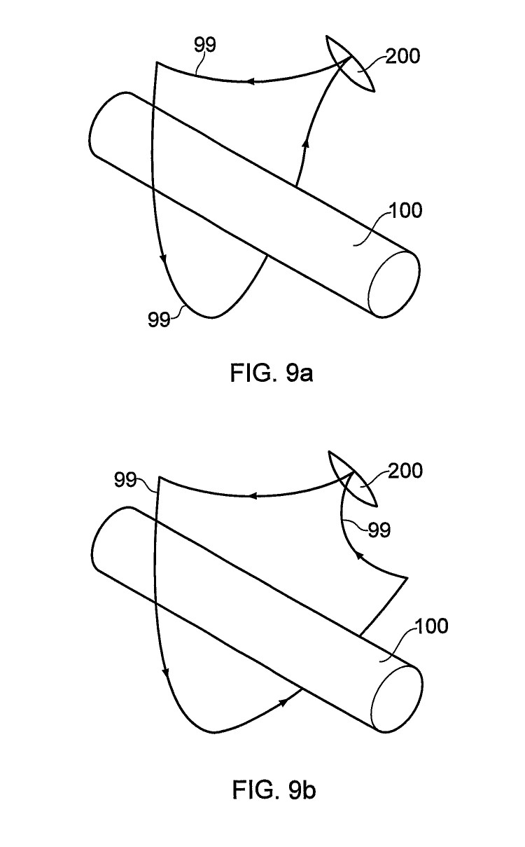

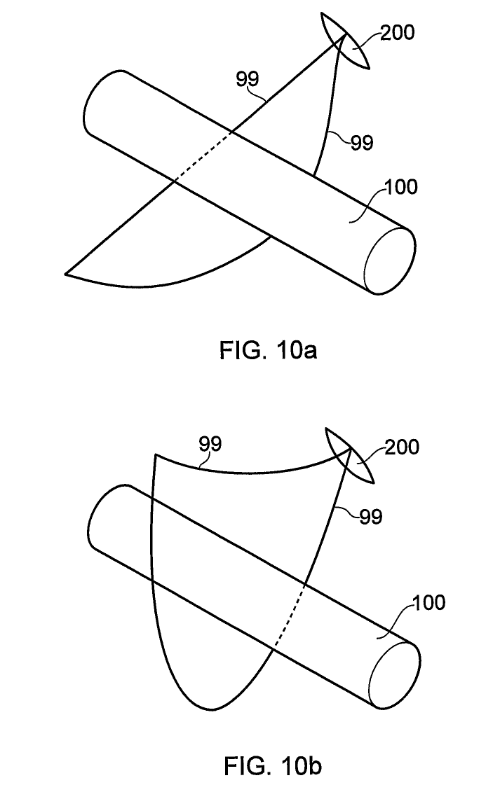

FIGS. 9a and 9b simply depict the path of suture 99 during the procedure. In FIGS. 10a and 10b a further embodiment of the invention is depicted wherein the suture is caused to pass through the vessel at least in one direction. This ensures the ensnarement of the vessel during the procedure.

If the needle is initially passed through the vein or other structure, the suture, now transfixing the vein, may act as a guidewire or lead through a separate guidewire to follow with other device components. Examples of other device components include components used to occlude or ablate the vein which may be used as an alternative or in addition to simple ligation by passage of the suture around the vein. Examples include devices for crimping, clipping or compressing the vein. The guidewire suture path through the vein may also be used to guide in one or more secondary devices to treat the vein. Such a procedure may be particularly useful in the treatment of veins which run generally parallel to the skin. Examples include the long and the short saphenous veins.

FIGS. 11a and 11b show the tightening of the suture around the vessel 100. Typically, the suture is knotted external the patient and the knot pushed down to the position shown in FIG. 11b.

The entire procedure is monitored using ultrasound imaging to guide the needle placement and confirm successful ligation/partial ligation/repair or any other form of modification required.

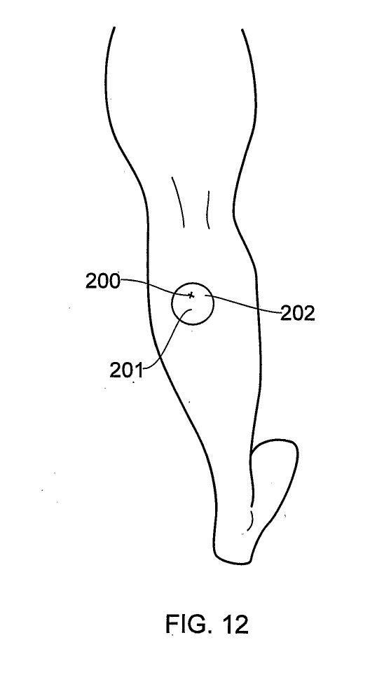

FIG. 12 depicts the calf region of an individual post treatment. Access portal 200 and exit sites 201 and 202 are small and no suturing is required. The procedure may, therefore, be carried out as an outpatient procedure under local anaesthetic.

### EXAMPLES

### Example 1

### Ligation of Incompetent Perforating Veins in the Leg Calf Region

The calf region is initially anaesthetised with local anaesthesia and the target vein carefully localised by physical examination of the leg and by ultrasound imaging.

A small incision of approximately 2 mm in length and 5 mm in depth is made about 1 cm from the target for initial placement of the needle assembly (primary access site). The first tissue piercing end is then directed to pass close to and either over or under the vein and then out of the skin approx 1 cm beyond the vein, retaining the second tissue piercing end, the shuttle member and suture under the skin. The first tissue piercing end of the needle, now external the skin is then held and used to draw the second tissue piercing end of the needle under the skin and beyond the target vein. The second end is then manipulated back around the other side of the target vein so that the suture now encircles it.

In most trials this process has been repeated a third time, so that the puncture sites form roughly a triangle with its points equidistant from the target vein, resulting in the suture passing close to the vein on three aspects and now allowing the suture to be drawn tight around the vein by finally bringing the whole needle assembly out through the primary access site and securing a knot at that point, under the skin. The primary access incision is small and is closed by simple placement of a tape or dressing, with no need for suture closure of the skin.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

1. A needle assembly comprising:
a curved shaft having a first tissue piercing end and a second tissue piercing end;
at least one shuttle member moveable relative to said shaft between said first and second tissue piercing ends;
said at least one shuttle member comprising at least one suture securing means to secure at least one suture to the shuttle member.

2. A needle assembly comprising:
a curved shaft having a first tissue piercing end and a second tissue piercing end;
at least one shuttle member moveable relative to said shaft between said first and second tissue piercing ends; and
at least one suture connected to said at least one shuttle member.

3. The needle assembly of claim 1 for use in ligating a vessel.

4. The needle assembly of claim 3 wherein said vessel comprises a vein.

5. The needle assembly of claim 1 wherein the curved shaft of the needle assembly includes a circular arc.

6. The needle assembly of claim 5 wherein the curvature of the shaft subtends an arc between 90° and 300°.

7. The needle assembly of claim 6 wherein the curvature of the shaft subtends an arc between 100° and 150°.

8. The needle assembly of claim 1 wherein the curvature of the shaft is non-circular.

9. The needle assembly of claim 1 wherein the shaft includes one or more non-curved sections.

10. The needle assembly of claim 1 wherein the cross-section of the curved shaft is circular.

11. The needle assembly of claim 1 wherein the cross section of the curved shaft is non-circular.

12. The needle assembly of claim 1 wherein said shuttle member is longitudinally moveable along at least a length of said curved shaft.

13. The needle assembly of claim 1 wherein the shuttle member is rotationally moveable relative to the curved shaft.

14. The needle assembly of claim 1 wherein the shuttle member is positioned external to the shaft.

15. The needle assembly of claim 1 including stop means to prevent the shuttle member moving beyond one or both ends of the shaft.

16. The needle assembly of claim 1 wherein the cross-sectional shape of the shuttle member corresponds to the cross-sectional shape of the curved shaft.

17. The needle assembly of claim 1 wherein the shuttle member includes radiopaque markers.

18. The needle assembly of claim 1 wherein the shuttle member comprises at least one aperture in the shuttle to receive a suture therein.

19. The needle assembly of claim 1 further comprising at least one tethering member to secure said suture to said shuttle member.

20. The needle assembly of claim 1 comprise a plurality of shuttle members.

21. The needle assembly of claim 1 wherein said shuttle member comprises a plurality of suture securing means.

22. The needle assembly of claim 1 wherein said first and second tissue piercing ends comprise trocar tips.

23. A method of ligating, repairing or modifying a target structure within an individual, comprising:
(a) providing a needle assembly comprising a curved shaft having a first tissue piercing end and a second tissue piercing end;
a shuttle member moveable relative to said curved shaft between said first and second tissue piercing ends;
said shuttle member comprising at least one suture securing means which secures at least, one suture to the shuttle member;
(b) at a primary access site in the skin, directing the first tissue piercing end of the curved shaft to a position adjacent to the target structure;
(c) moving said curved shaft in a first direction such that said first tissue piercing end and at least a leading portion of the suture passes superiorly to or through said target structure and to a location beyond said target structure;
(d) causing said first tissue piercing end to exit the skin at a first exit site remote from the primary access site and wherein the second tissue piercing end, the shuttle member and at least a portion of the at least one suture is retained subcutaneously;
(e) continuing movement of the curved shaft further in said first direction such that the second tissue piercing end is also positioned beyond said target structure;
(f) exerting pressure on said needle assembly to move the second tissue piercing end and the at least said leading portion of the suture in a second, different direction such that said second tissue piercing end and the at least leading portion of the suture passes inferiorly to or through said target structure;
(g) causing said second tissue piercing end to exit the skin at a second exit site remote from the primary access site and the first exit site wherein the first tissue piercing end, the shuttle member and at least a portion of the at least one suture is retained subcutaneously;
(h) securing at least the leading portion of the suture to a trailing portion of the suture.

24. A method of ligating, repairing or modifying a target structure within an individual, comprising:
(a) providing a needle assembly comprising a curved shaft having a first tissue piercing end and a second tissue piercing end;
a shuttle member moveable relative to said curved shaft between said first and second tissue piercing ends;
said shuttle member comprising at least one suture securing means which secures at least one suture to the shuttle member;
(b) at a primary access site in the skin, directing the first tissue piercing end of the curved shaft to a position adjacent to the target structure;
(c) moving said curved shaft in a first direction such that said first tissue piercing end and at least a leading portion of the suture passes inferiorly relative to said target structure and to a location beyond said target structure;
(d) causing said first tissue piercing end to exit the skin at a first exit site remote from the primary access site and wherein the second tissue piercing end, the shuttle member and at least a portion of the at least one suture is retained subcutaneously;
(e) continuing movement of the curved shaft further in said first direction, such that the second tissue piercing end is also positioned beyond said target structure;
(f) exerting pressure on said needle assembly to move the second tissue piercing end and the at least said leading portion of the suture in a second, different direction such that said second tissue piercing end and the at least leading portion of the suture passes superiorly relative to said target structure;
(g) causing said second tissue piercing end to exit the skin at a second exit site remote from the primary access site and the first exit site wherein the first tissue piercing end, the shuttle member and at least a portion of the at least one suture is retained subcutaneously;
(h) securing at least the leading portion of the suture to a trailing portion of the suture.

25. The method of claim 23 wherein step (h) comprises exerting pressure on the needle assembly to move the first tissue piercing end and the at least said leading portion of the suture in a third direction such that said first tissue piercing end and the at least leading portion of the suture pass through the primary access site.
